(19) United States
(12) Patent Application Publication
(10) Pub. No.: US 2023/0201840 A1
(43) Pub. Date: Jun. 29, 2023

(54) CONVECTIVE POLYMERASE CHAIN REACTION APPARATUS AND OPTICAL DETECTING METHOD THEREOF

(71) Applicant: Industrial Technology Research Institute, Hsinchu (TW)

(72) Inventors: Chun-Chuan Lin, Hsinchu City (TW); Hsiao-Yue Tsao, Hsinchu County (TW); Shih-Bin Luo, Hsinchu County (TW); Kuo-Hsing Wen, Hsinchu County (TW); Chien-Chih Kuo, Hsinchu City (TW); Ping-Jung Wu, Changhua County (TW); Ruey-Shyan Hong, Taoyuan City (TW); Ting-Hsuan Chen, Hsinchu County (TW)

(73) Assignee: Industrial Technology Research Institute, Hsinchu (TW)

(21) Appl. No.: 17/564,243

(22) Filed: Dec. 29, 2021

(51) Int. Cl. B01L 7/00; G01N 21/64; B01L 9/00

(52) U.S. Cl. CPC B01L 7/52; G01N 21/6486; B01L 9/527; G01N 2021/6419; G01N 2021/6421; C12Q 1/686

(57) ABSTRACT

A convective polymerase chain reaction apparatus and an optical detecting method thereof are provided. The optical detecting method includes the following steps. A substance in a tube to be tested is heated. At least two monochromatic lights are provided and are combined using a light combining element to irradiate the tube to be tested. At least two excited lights generated by exciting the substance in the tube to be tested by the at least two monochromatic lights are sensed.

### TECHNICAL FIELD

The disclosure relates to a reaction apparatus and a detecting method, and more particularly to a convective polymerase chain reaction apparatus and an optical detecting method thereof.

### DESCRIPTION OF RELATED ART

With the prevalence of various infectious diseases, rapid and accurate virus detecting methods are urgently needed to prevent and monitor a pandemic of infectious diseases. Viral nucleic acid detection using nucleic acid extraction and polymerase chain reaction may detect whether a subject's body contains viral gene fragments. The detecting method is highly sensitive and as long as there is a small amount of virus in the subject's body, the virus may be detected.

In a convective polymerase chain reaction, the principle of convection is adopted to directly heat the reaction vessel containing the liquid to be tested, so that a temperature gradient of the liquid to be tested is generated and convection occurs. By continuously changing the temperature of the liquid to be tested during the convection process, the convection of the liquid to be tested may amplify nucleic acids, which shortens the time required to change the temperature in a conventional method and has the advantages of simple structure and low cost of the testing apparatus. The currently adopted convective polymerase chain reaction apparatus uses a capillary test tube made of plastic or glass as a reaction vessel, and uses light beams to irradiate the test tube to excite the fluorescent reagent inside the tube in order to generate a fluorescent signal, and then uses a sensor to sense the fluorescent signal to monitor the polymerase chain reaction.

Polymerase chain reaction is a molecular biology technique used to amplify specific deoxyribonucleic acid (DNA) or ribonucleic acid (RNA) fragments. A typical polymerase chain reaction apparatus adopts a thermocycler to repeatedly heat up and cool down in order to repeat steps of a thermal cycle. Heat is gradually conducted into the reaction tube through the metal block, so that the DNA or RNA fragments undergo three different types of temperature cycles, such as denaturation (95° C.), annealing (45° C.-65° C.), and extension (72° C.) under different temperatures. However, in the case where there is more than one type of target virus to be detected, each apparatus can only detect one type of virus for one tube to be tested, corresponding screening needs to be performed according to the number of types of target viruses to obtain the same number of tubes to be tested, which increases the burden on the subject.

### SUMMARY

The disclosure provides a convective polymerase chain reaction apparatus and an optical detecting method thereof, which may detect multiple targets at the same time, thereby reducing the total number of specimens required.

The convective polymerase chain reaction apparatus of the disclosure includes a base, a heating element, a light combining element, at least two light emitting elements, and at least two light sensors. A number of light sensors corresponds to a number of light emitting elements.

The base is suitable for carrying a tube to be tested. The heating element is installed on the base to heat the tube to be tested through the base. The light combining element includes a first plate portion, a second plate portion, a third plate portion, and a fourth plate portion connected to one another. The first plate portion and the second plate portion define a light exit area. The second plate portion and the third plate portion define a first light incident area. The third plate portion and the fourth plate portion define a second light incident area. The first plate portion and the fourth plate portion define a third light incident area. The light emitting elements are at least two of a first light emitting element, a second light emitting element, and a third light emitting element. The first light emitting element is suitable for providing a first monochromatic light toward the first light incident area. The second light emitting element is suitable for providing a second monochromatic light toward the second light incident area. The third light emitting element is suitable for providing a third monochromatic light toward the third light incident area. The first plate portion is suitable for reflecting the first monochromatic light and allowing the second monochromatic light and the third monochromatic light to pass through. The second plate portion is suitable for reflecting the third monochromatic light and allowing the first monochromatic light and the second monochromatic light to pass through. The third plate portion is suitable for reflecting the first monochromatic light and allowing the second monochromatic light to pass through. The fourth plate portion is suitable for reflecting the third monochromatic light and allowing the second monochromatic light to pass through. At least two of the first monochromatic light, the second monochromatic light, and the third monochromatic light enter the light exit area and emit light toward the base to irradiate the tube to be tested. At least two of the first light sensor, the second light sensor, and the third light sensor are installed on the base. The light sensors are installed on the base and are at least two of a first light sensor, a second light sensor, and a third light sensor. The first light sensor is suitable for receiving a first excited light generated by exciting a substance in the tube to be tested with the first monochromatic light. The second light sensor is suitable for receiving a second excited light generated by exciting the substance in the tube to be tested with the second monochromatic light. The third light sensor is suitable for receiving a third excited light generated by exciting the substance in the tube to be tested with the third monochromatic light.

The optical detecting method in the disclosure uses the convective polymerase chain reaction apparatus for detection, and the method includes the following steps. The substance in the tube to be tested is heated. At least two of the first monochromatic light, the second monochromatic light, and the third monochromatic light are provided and are combined using the light combining element to irradiate the tube to be tested. At least two of the first excited light generated by exciting the substance in the tube to be tested with the first monochromatic light, the second excited light generated by exciting the substance in the tube to be tested with the second monochromatic light, and the third excited light generated by exciting the substance in the tube to be tested with the third monochromatic light are sensed.

Based on the above, in the convective polymerase chain reaction apparatus and the optical detecting method thereof of the disclosure, at least two monochromatic lights may be detected at the same time, thereby reducing the number of specimens used.

### BRIEF DESCRIPTION OF THE DRAWINGS

FIG. 1 is a three-dimensional cross-sectional view of a convective polymerase chain reaction apparatus according to an embodiment of the disclosure.

FIG. 2 is a partial top schematic view of the convective polymerase chain reaction apparatus of FIG. 1.

FIG. 3 is a schematic view illustrating a function of a condenser unit of the convective polymerase chain reaction apparatus of FIG. 1.

FIG. 4 is a schematic view of the condenser unit of the convective polymerase chain reaction apparatus of FIG. 3 after an adjustment.

FIG. 5 is an exploded schematic view of a light combining element of the convective polymerase chain reaction apparatus of FIG. 1.

FIG. 6 is a partial top schematic view of the light combining element of FIG. 5.

### DETAILED DESCRIPTION OF DISCLOSED EMBODIMENTS

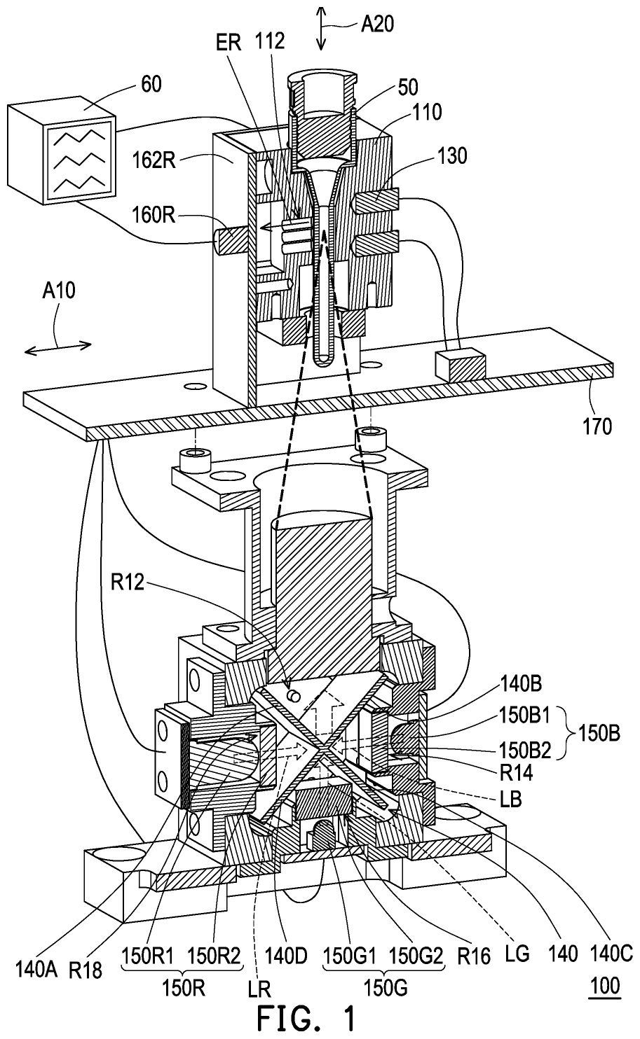

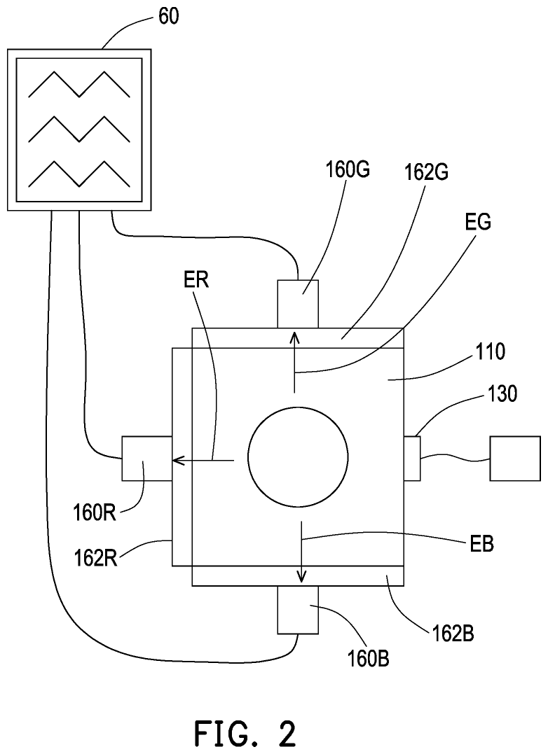

FIG. 1 is a three-dimensional cross-sectional view of a convective polymerase chain reaction apparatus according to an embodiment of the disclosure. FIG. 2 is a partial top schematic view of the convective polymerase chain reaction apparatus of FIG. 1, wherein some elements of the convective polymerase chain reaction apparatus are omitted in FIG. 2. Referring to FIGS. 1 and 2, a convective polymerase chain reaction apparatus 100 of the embodiment is suitable for performing convective polymerase chain reaction, which amplifies specific deoxyribonucleic acid or ribonucleic acid fragments for virus detection, but the purpose of usage of the convective polymerase chain reaction apparatus 100 of the disclosure is not limited.

The convective polymerase chain reaction apparatus 100 includes a base 110, a heating element 130, a light combining element 140, at least two light emitting elements, and at least two light sensors. The number of light sensors is set corresponding to the number of light emitting elements. As shown in FIG. 1, the light emitting elements may be at least two of a first light emitting element 150B, a second light emitting element 150G, and a third light emitting element 150R; the light sensors may be at least two of a first light sensor 160B, a second light sensor 160G, and a third light sensor 160R. In other words, the light emitting elements and the light sensors in the convective polymerase chain reaction apparatus 100 of the embodiment may include only the first light emitting element 150B, the second light emitting element 150G, the first light sensor 160B, and the second light sensor 160G; only the third light emitting element 150R, the second light emitting element 150G, the third light sensor 160R, and the second light sensor 160G; only the first light emitting element 150B, the third light emitting element 150R, the first light sensor 160B, and the third light sensor 160R; or the first light emitting element 150B, the second light emitting element 150G, the third light emitting element 150R, the first light sensor 160B, the second light sensor 160G, and the third light sensor 160R at the same time. Hereinafter, the convective polymerase chain reaction apparatus 100 will include the first light emitting element 150B, the second light emitting element 150G, the third light emitting element 150R, the first light sensor 160B, the second light sensor 160G, and the third light sensor 160R at the same time as the example for the description.

The base 110 is suitable for carrying a tube to be tested 50. The heating element 130 is installed on the base 110 to heat the tube to be tested 50 through the base 110. The light combining element 140 includes a first plate portion 140A, a second plate portion 140B, a third plate portion 140C, and a fourth plate portion 140D connected to one another. Specifically, the first plate portion 140A, the second plate portion 140B, the third plate portion 140C, and the fourth plate portion 140D are each connected to the other three through a surface to form an X-shaped structure. The first plate portion 140A and the second plate portion 140B define a light exit area R12. The second plate portion 140B and the third plate portion 140C define a first light incident area R14. The third plate portion 140C and the fourth plate portion 140D define a second light incident area R16. The first plate portion 140A and the fourth plate portion 140D define a third light incident area R18.

The first light emitting element 150B is suitable for providing a first monochromatic light LB toward the first light incident area R14. The second light emitting element 150G is suitable for providing a second monochromatic light LG toward the second light incident area R16. The third light emitting element 15OR is suitable for providing a third monochromatic light LR toward the third light incident area R18. The first plate portion 140A is suitable for reflecting the first monochromatic light LB and allowing the second monochromatic light LG and the third monochromatic light LR to pass through. The second plate portion 140B is suitable for reflecting the third monochromatic light LR and allowing the first monochromatic light LB and the second monochromatic light LG to pass through. The third plate portion 140C is suitable for reflecting the first monochromatic light LB and allowing the second monochromatic light LG to pass through. The fourth plate portion 140D is suitable for reflecting the third monochromatic light LR and allowing the second monochromatic light LG to pass through. Therefore, the first plate portion 140A and the third plate portion 140C may be dichroic mirrors with the same optical characteristic, and the second plate portion 140B and the fourth plate portion 140D may be dichroic mirrors with another optical characteristic. At least two of the first monochromatic light LB, the second monochromatic light LG, and the third monochromatic light LR enter the light combining element 140 through at least two of the corresponding first light incident area R14, second light incident area R16, and third light incident area R18, enter the light exit area R12 through the optical effects (such as reflection and penetration) of the first plate portion 140A, the second plate portion 140B, the third plate portion 140C, and the fourth plate portion 140D, and then exit toward the base 110 to irradiate the tube to be tested 50.

Based on the X-shaped structure design of the light combining element 140, the first monochromatic light LB may directly pass through the second plate portion 140B after entering the first light incident area R14 to reach the first plate portion 140A, and enter the light exit area R12 after being reflected by the first plate portion 140A or may also pass through the second plate portion 140B after being reflected by the third plate portion 140C to enter the light exit area R12. The second monochromatic light LG may directly pass through the third plate portion 140C and the second plate portion 140B after entering the second light incident area R16 to enter the light exit area R12 or may directly pass through the fourth plate portion 140D and the first plate portion 140A to enter the light exit area R12. The third monochromatic light LR may directly pass through the first plate portion 140A after entering the third light incident area R18 to reach the second plate portion 140B, and enter the light exit area R12 after being reflected by the second plate portion 140B or may also pass through the first plate portion 140A after being reflected by the fourth plate portion 140D to enter the light exit area R12. Therefore, the first monochromatic light LB, the second monochromatic light LG, and the third monochromatic light LR entering different light incident areas all enter the light exit area R12 and exit toward the base 110 to irradiate the tube to be tested 50.

At least two of the first light sensor 160B, the second light sensor 160G, and the third light sensor 160R are installed on the base 110. The first light sensor 160B is suitable for receiving a first excited light EB generated by exciting a substance in the tube to be tested 50 with the first monochromatic light LB. The second light sensor 160G is suitable for receiving a second excited light EG generated by exciting the substance in the tube to be tested 50 with the second monochromatic light LG. The third light sensor 160R is suitable for receiving a third excited light ER generated by exciting the substance in the tube to be tested 50 with the third monochromatic light LR.

The optical detecting method of the embodiment uses the convective polymerase chain reaction apparatus 100 for detection, and the method includes the following steps. The substance in the tube to be tested 50 is heated to generate a temperature gradient of the substance in the tube to be tested 50 and cause convection, thereby generating nucleic acid amplification. In addition, at least two of the first monochromatic light LB, the second monochromatic light LG, and the third monochromatic light LR are provided and are combined using the light combining element 140 to irradiate the tube to be tested 50. Then, at least two of the first excited light EB generated by exciting the substance in the tube to be tested 50 with the first monochromatic light LB, the second excited light EG generated by exciting the substance in the tube to be tested 50 with the second monochromatic light LG, and the third excited light ER generated by exciting the substance in the tube to be tested 50 with the third monochromatic light LR are sensed.

In the convective polymerase chain reaction apparatus 100 of the embodiment, since the light combining element 140 is adopted, the tube to be tested 50 may be irradiated by multiple monochromatic light at the same time, so that different substances in the tube to be tested 50 are excited by different monochromatic light to generate different excited lights. As a result, multiple substances in the tube to be tested 50 may be detected at the same time, thereby reducing the number of times of screening to reduce the burden on the subject.

In addition, the light combining element 140 of the embodiment has an X-shaped structure composed of the first plate portion 140A, the second plate portion 140B, the third plate portion 140C, and the fourth plate portion 140D, which is lighter in comparison to a solid cube type light combining element and may also reduce the problems caused by total reflection. In addition, since the light combining element 140 is separated from the base 110, the first light emitting element 150B, the second light emitting element 150G, and the third light emitting element 15OR beside the light combining element 140 will also be away from the heating element 130 beside the base 110, such that the first light emitting element 150B, the second light emitting element 150G, and the third light emitting element 15OR may be prevented from being heated and changing the light emitting characteristics.

The base 110 of the embodiment is suitable for carrying the tube to be tested 50 in the center thereof. In detail, the tube to be tested 50 is configured in the base 110 in a direction perpendicular to the horizontal plane, and the first light sensor 160B, the second light sensor 160G, and the third light sensor 160R are respectively configured on three sides of the base 110, and the heating element 130 is located on the other side of the base 110. In the embodiment, the four sides of the base 110 that are configured with the first light sensor 160B, the second light sensor 160G, the third light sensor 160R, and the heating element 130 surround the tube to be tested 50. In other words, the first light sensor 160B, the second light sensor 160G, the third light sensor 160R, and the heating element 13 are configured around the tube to be tested 50 through the base 110. After the first light sensor 160B, the second light sensor 160G, and the third light sensor 160R respectively receive the first excited light EB, the second excited light EG, and the third excited light ER, signals are transmitted to a spectrometer 60 for analysis to confirm whether there is a substance set as a detection target in the tube to be tested 50.

In the embodiment, the convective polymerase chain reaction apparatus 100 further includes at least two filter elements. The filter elements may be at least two of a first filter element 162B, a second filter element 162G, and a third filter element 162R. The number of filter elements is set corresponding to the number of light sensors. For example, in the case where the convective polymerase chain reaction apparatus 100 contains the first light sensor 160B, the first filter element 162B may be correspondingly configured, in the case where the convective polymerase chain reaction apparatus 100 contains the second light sensor 160G, the second filter element 162G may be correspondingly configured, and in case where the convective polymerase chain reaction apparatus 100 contains the third light sensor 160R, the third filter element 162R may be correspondingly configured. Specifically, the first filter element 162B is configured between the first light sensor 160B and the base 110. The second filter element 162G is configured between the second light sensor 160G and the base 110. The third filter element 162R is configured between the third light sensor 160R and the base 110. The filter element may filter the excited light before sensing. For example, the first filter element 162B may further filter out light other than the first excited light EB to prevent the first light sensor 160B from receiving the second excited light EG and the third excited light ER and reducing the sensing accuracy. The second filter element 162G may further filter out light other than the second excited light EG to prevent the second light sensor 160G from receiving the first excited light EB and the third excited light ER and reducing the sensing accuracy. The third filter element 162R may further filter out light other than the third excited light ER to prevent the third light sensor 160R from receiving the first excited light EB and the second excited light EG and reducing the sensing accuracy.

The convective polymerase chain reaction apparatus 100 of the embodiment may further include a circuit board 170. The base 110 and the heating element 130 may be configured on the circuit board 170, and the light combining element 140 may be configured under the circuit board 170. The first light emitting element 150B, the second light emitting element 150G, and the third light emitting element 15OR may be electrically connected to the circuit board 170.

In the embodiment, the base 110 has multiple elongated through holes 112. A long axis A10 of the elongated through hole 112 is perpendicular to a long axis A20 of the tube to be tested 50 carried in the base 110. The elongated through holes 112 may be correspondingly and respectively communicated with the first light sensor 160B, the second light sensor 160G, and the third light sensor 160R, so that the first excited light EB, the second excited light EG, and the third excited light ER may pass through. The elongated through hole 112 has a collimating effect on the first excited light EB, the second excited light EG, and the third excited light ER. The elongated through hole 112 is perpendicular to the tube to be tested 50, which is equivalent to being perpendicular to the light incident direction when the three monochromatic lights irradiate the tube to be tested 50. Therefore, the probability of the three monochromatic lights entering the three sensors through the elongated through holes 112 may be greatly reduced, thereby reducing interference and improving the sensing accuracy. In addition, in order to increase the amount of incident light, multiple corresponding elongated through holes 112 may be configured for each sensor, so that the excited light is collimated through the elongated through holes 112 before being sensed. As shown in FIG. 1, in the embodiment, three elongated through holes 112 arranged along the long axis A20 of the tube to be tested 50 carried in the base 110 are correspondingly communicated with the third sensor 160R, so that the first excited light EB, the second excited light EG, and the third excited light ER may pass through, which are transmitted to the third sensor 160R after noise is being filtered out by the third filter element 162R.

In the embodiment, each light emitting element may include a light emitting diode and a filter. Specifically, the first light emitting element 150B includes a light emitting diode 150B1 and a filter 150B2. The filter 150B2 is configured between the light emitting diode 150B1 and the light combining element 140. The filter 150B2 may ensure that the first monochromatic light LB irradiated to the tube to be tested 50 has a preset wavelength, so as to improve the sensing accuracy. Similarly, the second light emitting element 150G includes a light emitting diode 150G1 and a filter 150G2. The filter 150G2 is configured between the light emitting diode 150G1 and the light combining element 140. The third light emitting element 150R includes a light emitting diode 150R1 and a filter 150R2. The filter 150R2 is configured between the light emitting diode 150R1 and the light combining element 140. The light emitting diode may be an organic or inorganic light emitting diode.

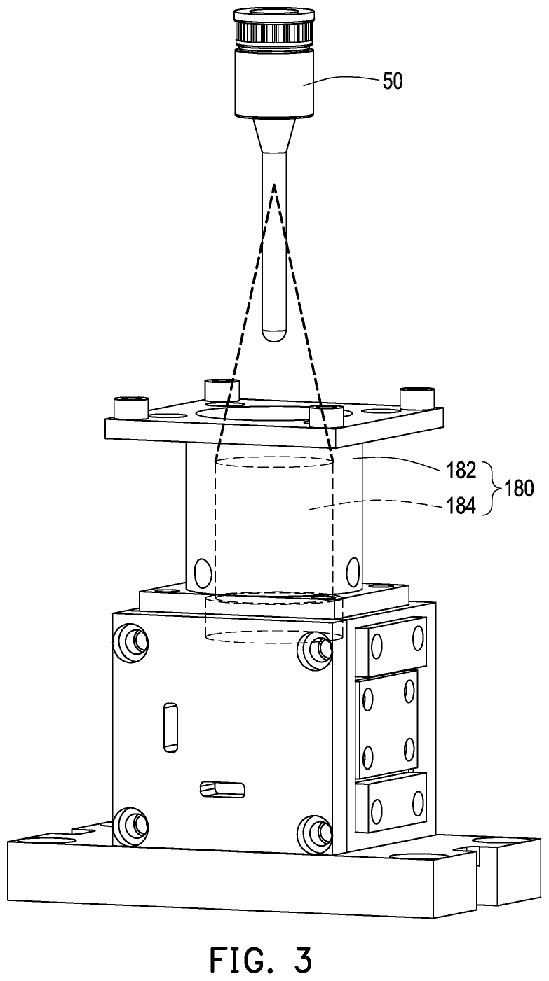

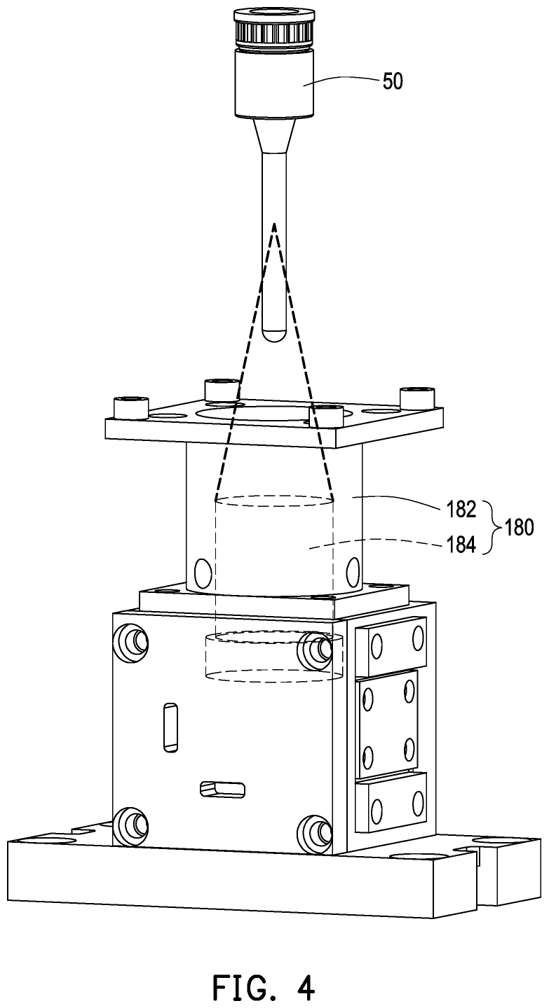

FIG. 3 is a schematic view illustrating a function of a condenser unit of the convective polymerase chain reaction apparatus of FIG. 1. Referring to FIG. 3, in the embodiment, the convective polymerase chain reaction apparatus 100 further includes a condenser unit 180, which is configured between the light exit area R12 of the light combining element 140 and the base 110, is suitable for focusing at least two of the first monochromatic light LB, the second monochromatic light LG, and the third monochromatic light LR onto the tube to be tested 50, and is suitable for adjusting the focus position. FIG. 4 is a schematic view of the condenser unit of the convective polymerase chain reaction apparatus of FIG. 3 after an adjustment, and some elements of the convective polymerase chain reaction apparatus are omitted in FIGS. 3 and 4. In FIG. 3, the condenser unit 180 focuses the combined light of multiple monochromatic lights (that is, at least two of the first monochromatic light LB, the second monochromatic light LG, and the third monochromatic light LR) on a higher position on the tube to be tested 50. On the other hand, in FIG. 4, the condenser unit 180 focuses the combined light of multiple monochromatic lights on a lower position on the tube to be tested 50. That is, adjusting the condenser unit 180 may adjust the focus position. In the process of adjusting the focus position, a good focus adjustment position may be found, that is, the position where the sensing effect of the three sensors is good.

In addition, the condenser unit 180 allows the focus position to avoid the bottom of the tube to be tested 50. Since impurities are easily deposited on the bottom of the tube to be tested 50, allowing the focus position to avoid the bottom of the tube to be tested 50 may also reduce interference and improve the sensing accuracy. The focus position may be adjusted to be located at the upper half of the substance in the tube to be tested 50 to be away from the bottom of the tube to be tested 50. In the embodiment, the condenser unit 180 includes a lens barrel 182 and a condenser lens 184. The condenser lens 184 is movably configured in the lens barrel 182. The focus position may be adjusted by moving the relative positions of the lens barrel 182 and the condenser lens 184.

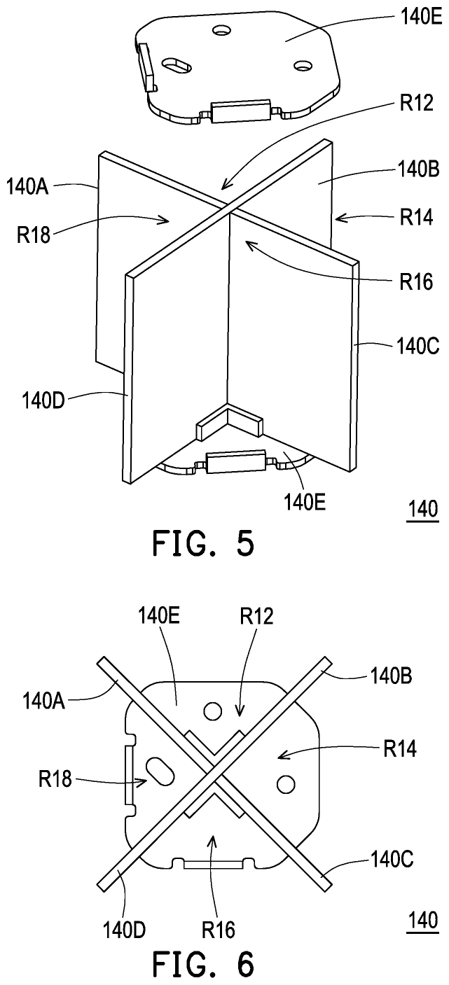

FIG. 5 is an exploded schematic view of a light combining element of the convective polymerase chain reaction apparatus of FIG. 1. FIG. 6 is a partial top schematic view of the light combining element of FIG. 5. Referring to FIGS. 5 and 6, in the embodiment, the first plate portion 140A, the second plate portion 140B, the third plate portion 140C, and the fourth plate portion 140D connected to one another form an X-shaped structure. For example, included angles between the first plate portion 140A, the second plate portion 140B, the third plate portion 140C, and the fourth plate portion 140D are all 90 degrees, but the disclosure is not limited thereto. In the embodiment, the light combining element 140 further includes two fixing seats 140E. The first plate portion 140A, the second plate portion 140B, the third plate portion 140C, and the fourth plate portion 140D are fixed between the two fixing seats 140E.

For example, the first plate portion 140A and the third plate portion 140C may have the same optical characteristics, that is, both the first plate portion 140A and the third plate portion 140C reflect the first monochromatic light LB and allow the second monochromatic light LG and the third monochromatic light LR to pass through. The second plate portion 140B and the fourth plate portion 140D may have the same optical characteristics, that is, both the second plate portion 140B and the fourth plate portion 140D reflect the third monochromatic light LR and allow the second monochromatic light LG and the first monochromatic light LB to pass through. Therefore, the first plate portion 140A and the third plate portion 140C of the embodiment may be the same optical element, while the second plate portion 140B and the fourth plate portion 140D are two separate optical elements. Such design facilitates assembly, but the disclosure is not limited thereto.

In the embodiment, the wavelength of the first monochromatic light LB is, for example, between 451.5 nm and 486.5 nm, the wavelength of the second monochromatic light LG is, for example, between 507 nm and 527 nm, and the wavelength of the third monochromatic light LR is, for example, between 612 nm and 644 nm. In another embodiment, the wavelength of the first monochromatic light LB is, for example, between 506 nm and 534 nm, the wavelength of the second monochromatic light LG is, for example, between 543 nm and 566 nm, and the wavelength of the third monochromatic light LR is, for example, between 672 nm and 696 nm. Meanwhile, the first excited light EB generated by irradiating the substance in the tube to be tested 50 according to the monochromatic light range of the above interval is, for example, suitable for detecting the influenza A virus, the second excited light EG is, for example, suitable for detecting the influenza B virus, and the third excited light ER is, for example, suitable for detecting the Covid-19 virus.

In summary, in the convective polymerase chain reaction apparatus and the optical detecting method thereof of the disclosure, since the light combining element is used, multiple monochromatic lights may be combined for detection, and multiple targets may be detected at the same time, thereby reducing the number of specimens used to reduce the burden on the subject.

What is claimed is:

1. A convective polymerase chain reaction apparatus, comprising:
a base, suitable for carrying a tube to be tested;
a heating element, installed on the base to heat the tube to be tested through the base;
a light combining element, comprising a first plate portion, a second plate portion, a third plate portion, and a fourth plate portion connected to one another, wherein the first plate portion and the second plate portion define a light exit area, the second plate portion and the third plate portion define a first light incident area, the third plate portion and the fourth plate portion define a second light incident area, the first plate portion and the fourth plate portion define a third light incident area;
at least two light emitting elements; and
at least two light sensors,
wherein a number of the at least two light sensors corresponds to a number of the at least two light emitting elements,
the light emitting elements are at least two of a first light emitting element, a second light emitting element, and a third light emitting element, the first light emitting element is suitable for providing a first monochromatic light toward the first light incident area, the second light emitting element is suitable for providing a second monochromatic light toward the second light incident area, the third light emitting element is suitable for providing a third monochromatic light toward the third light incident area, the first plate portion is suitable for reflecting the first monochromatic light and allowing the second monochromatic light and the third monochromatic light to pass through, the second plate portion is suitable for reflecting the third monochromatic light and allowing the first monochromatic light and the second monochromatic light to pass through, the third plate portion is suitable for reflecting the first monochromatic light and allowing the second monochromatic light to pass through, the fourth plate portion is suitable for reflecting the third monochromatic light and allowing the second monochromatic light to pass through, at least two of the first monochromatic light, the second monochromatic light, and the third monochromatic light enter the light exit area and exit toward the base to irradiate the tube to be tested; and
at least two of a first light sensor, a second light sensor, and a third light sensor are installed on the base, wherein the first light sensor is suitable for receiving a first excited light generated by exciting a substance in the tube to be tested with the first monochromatic light, the second light sensor is suitable for receiving a second excited light generated by exciting the substance in the tube to be tested with the second monochromatic light, the third light sensor is suitable for receiving a third excited light generated by exciting the substance in the tube to be tested with the third monochromatic light.

2. The convective polymerase chain reaction apparatus according to claim 1, wherein the first plate portion, the second plate portion, the third plate portion, and the fourth plate portion are each connected to other three through a surface to form an X-shaped structure.

3. The convective polymerase chain reaction apparatus according to claim 1, further comprising a condenser unit, configured between the light exit area of the light combining element and the base, is suitable for focusing at least two of the first monochromatic light, the second monochromatic light, and the third monochromatic light onto the tube to be tested, and is suitable for adjusting a focus position.

4. The convective polymerase chain reaction apparatus according to claim 3, wherein the condenser unit comprises a lens barrel and a condenser lens, the condenser lens is movably configured in the lens barrel.

5. The convective polymerase chain reaction apparatus according to claim 1, wherein the light combining element further comprises two fixing seats, the first plate portion, the second plate portion, the third plate portion, and the fourth plate portion are fixed between the fixing seats.

6. The convective polymerase chain reaction apparatus according to claim 1, further comprising at least two filter elements, a number of the at least two filter elements is set corresponding to the number of the at least two light sensors, the filter elements are at least two of a first filter element, a second filter element, and a third filter element, wherein the first filter element is configured between the first light sensor and the base, the second filter element is configured between the second light sensor and the base, the third filter element is configured between the third light sensor and the base.

7. The convective polymerase chain reaction apparatus according to claim 1, wherein the base has a plurality of elongated through holes, long axes of the elongated through holes are perpendicular to a long axis of the tube to be tested carried in the base and are respectively and correspondingly communicated with the first light sensor, the second light sensor, and the third light sensor.

8. The convective polymerase chain reaction apparatus according to claim 1, wherein each of the light emitting elements comprises a light emitting diode and a filter, the filter is configured between the light emitting diode and the light combining element.

9. An optical detecting method, using the convective polymerase chain reaction apparatus according to claim 1 for detection, comprising:
heating the substance in the tube to be tested;
providing at least two of the first monochromatic light, the second monochromatic light, and the third monochromatic light, and combining the at least two of the first monochromatic light, the second monochromatic light, and the third monochromatic light using the light combining element to irradiate the tube to be tested; and
sensing at least two of the first excited light generated by exciting the substance in the tube to be tested with the first monochromatic light, the second excited light generated by exciting the substance in the tube to be tested with the second monochromatic light, and the third excited light generated by exciting the substance in the tube to be tested with the third monochromatic light.

10. The optical detecting method according to claim 9, further comprising focusing a combined light of at least two of the first monochromatic light, the second monochromatic light, and the third monochromatic light onto the tube to be tested using a condenser unit, and adjusting the condenser unit to adjust a focus position.

11. The optical detecting method according to claim 9, further comprising filtering before sensing at least two of the first excited light, the second excited light, and the third excited light using at least two filter elements.

12. The optical detecting method according to claim 9, further comprising collimating before sensing at least two of the first excited light, the second excited light, and the third excited light using a plurality of elongated through holes.
